(19) United States
(12) Patent Application Publication
(10) Pub. No.: US 2021/0063317 A1
(43) Pub. Date: Mar. 4, 2021

(54) DETECTION OF DEFECTS ON METAL SURFACES

(71) Applicant: THE BOARD OF TRUSTEES OF THE UNIVERSITY OF ILLINOIS, Urbana, IL (US)

(72) Inventors: Shripriya Darshini Poduri, Aloha, OR (US); Michael A. Stroscio, Wilmette, IL (US); Mitra Dutta, Wilmette, IL (US); Richard S. Hill, Las Vegas, NV (US)

(21) Appl. No.: 16/983,511

(22) Filed: Aug. 3, 2020

Related U.S. Application Data
(60) Provisional application No. 62/894,252, filed on Aug. 30, 2019.

(51) Int. Cl. G01N 21/91; G01N 21/64; G01N 33/20; G01N 1/30; G01N 21/95; B08B 7/00; B08B 1/00

(52) U.S. Cl. CPC G01N 21/91; G01N 21/6456; G01N 33/20; B08B 1/006; G01N 21/95; B08B 7/0071; G01N 1/30

(57) ABSTRACT

In one aspect, the disclosure relates to the detection of defects on metal surfaces. In accordance with the purpose(s) of the present disclosure, as embodied and broadly described herein, the disclosure, in one aspect, relates to methods for using functionalized CdSe/ZnS quantum dots to detect damage on metal surfaces including, but not limited to, copper surfaces such as those found in passive components of electronic devices. Also disclosed herein are methods for removing bound quantum dots from metal surfaces. This abstract is intended as a scanning tool for purposes of searching in the particular art and is not intended to be limiting of the present disclosure.

### CROSS-REFERENCE TO RELATED APPLICATIONS

This application claims benefit of U.S. Provisional Application No. 62/894,252, filed Aug. 30, 2019, which is hereby incorporated herein by reference in its entirety.

### BACKGROUND

Growth in fields such as consumer electronics, data processing, telecommunications, military technology, the aerospace industry, the automotive industry, robotics and automation, and healthcare has driven corresponding increases in the market for passive components including, but not limited to, interconnects. Growth in these industries is expected to continue to increase in the coming years as more industrial processes are automated and more data is transferred via personal electronic devices.

Hardware failures are responsible for nearly three-fourths of network downtime. The cost of an unplanned data center outage can exceed $10,000 per minute for organizations that depend on service delivery such as, for example, telecommunications providers and e-commerce companies. Among consumer electronics, a significant number of laptop and/or desktop computers, LCD televisions, and plasma televisions fail by their fourth year, and warranty service for failures in home video game consoles represent a significant cost for their manufacturers. Modern automobiles incorporate significantly more electronic components than in past decades, as well.

Metal interconnects are used to improve performance of silicon integrated circuits such as those found in the devices and systems discussed previously. These interconnects can reduce propagation delays as well as power consumption. Previous technologies have been based on aluminum interconnects, but copper is a better conductor than aluminum and presents other advantages as well. For example, interconnects can have narrower dimensions when fabricated from copper, and the energy requirements for passing electricity through copper interconnects are lower.

The material properties of metal interconnects have a strong influence on the lifespan of the interconnects as well as on the lifespan of devices incorporating the interconnects. These characteristics are predominantly related to composition of the metal alloy from which the interconnect is formed as well as its dimensions. Shape, crystallographic orientation, procedures for deposition, heat treatment, and the nature of current sources also affect the durability of interconnects.

In microelectronics (that is, systems with submicron characteristic dimensions), electromigration becomes a problem. Electromigration occurs when the momentum of a moving electron is transferred to a nearby activated ion, causing the ion to move from its original position (see FIG. 1). Over time, this force displaces a significant number of atoms from their original positions, resulting in gaps in the conducting material and preventing the flow of electricity. In small dimension interconnect conductors, this is known as a void or an internal failure open circuit. Understanding the locations and dimensions of surface features such as voids, as well as nanoscale features, cracks, and other defects in copper interconnects is vital to failure analysis in microelectronics production.

Previous attempts to detect the locations of nano-sized pores and cavities in semiconductor wafers and/or metal surfaces have included X-ray detection, fluorescence detection, and optical microscopy. However, these methods are not ideal. For example, large numbers of defects and/or surface patterning may make optical inspection difficult. X-ray methods require expensive equipment and make use of ionizing radiation, which can present human health hazards.

Despite advances in visualization and characterization of voids in copper interconnects, there is still a scarcity of detection methods that are facile, efficacious, safe, and cost-effective. The present disclosure addresses these needs.

### SUMMARY

In accordance with the purpose(s) of the present disclosure, as embodied and broadly described herein, the disclosure, in one aspect, relates to methods for using functionalized CdSe/ZnS quantum dots to detect damage on metal surfaces including, but not limited to, copper surfaces such as those found in passive components of electronic devices. Also disclosed herein are methods for removing bound quantum dots from metal surfaces.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a schematic illustrating the process of electromigration in copper microelectronic components.

FIG. 2 shows a silicon wafer with copper contacts.

FIG. 3A and 3B show images of copper microelectronic components before the addition of quantum dots. In FIG. 3B, several voids can be seen in the copper surfaces. Magnification in both figures is 4×.

FIGS. 4A to 4D show images of various microelectronic components after drop casting of carboxyl-functionalized quantum dots over the components under normal lighting conditions (left panels) and using a 655 nm filter (right panels). In each instance, the components have been washed with deionized water after drop casting, indicating binding of the quantum dots to the copper surfaces. Magnification in FIGS. 4A-4C is 4× while magnification in FIG. 4D is 50×.

FIG. 5 shows SEM micrographs of copper surfaces that have been anodized for 600 s (left panel), 900 s (middle panel), and 1500 s (right panel).

FIG. 6 shows an anodized copper surface that has been drop cast with carboxyl-functionalized quantum dots. Top left panel: white light image under 4× magnification. Top right image: 4× magnification using a 655 nm filter. Bottom left image: white light image of the same area under 50× magnification. Bottom right image: 50× magnification of the same area using a 655 nm filter.

FIG. 7 shows an AFM micrograph of a copper surface with quantum dots bound to it.

FIG. 8 shows a copper surface where the left half has been anodized and the right half was masked to prevent anodization. Left panel: white light image; right panel: same area under 655 nm filter. Carboxyl-functionalized quantum dots were drop cast on the surface and only bound to the nanoporous left side of the surface, while they washed off the nonporous copper surface.

FIGS. 9A and 9B show that amino-functionalized quantum dots do not bind to anodized copper surfaces. FIG. 9A shows a copper surface after drop casting of amino-functionalized quantum dots under a 655 nm filter (left panel) and under white light (right panel). FIG. 9B (left panel) is a white light image where amino-functionalized quantum dots have been drop cast and the surface washed with deionized water contrasted with a red fluorescence (655 nm) image of the same area under 50× magnification (right panel) showing no binding of amino-functionalized quantum dots.

FIG. 10 shows additional views of an anodized copper surface bound to carboxy-functionalized quantum dots under white light (top row) and a 655 nm filter (bottom row) at 50× magnification, before washing (left column) and after washing (right column). Even after washing, quantum dots were observed bound to approximately the same areas.

FIG. 11 shows the copper surface under a 655 nm filter (left panel) and a white light image of the same (right panel) with bound carboxy-functionalized quantum dots after four washings with deionized water. Quantum dots were observed bound to the same areas. Magnification is 50× in both images.

FIGS. 12A and 12B show that bound quantum dots can be removed from copper surfaces with heating and wiping. FIG. 12A shows an anodized copper surface that was heated to 200 ° C. for 30 minutes, cooled, and wiped with a lint-free wipe to remove bound quantum dots. Left panel: white light image; right panel: fluorescence image under 655 nm filter showing that no quantum dots remain after heating. Magnification is 4×. FIG. 12B shows the same surface at 50× magnification.

FIGS. 13A and 13B show additional views demonstrating that masking copper during the anodization process prevents pore formation and thus prevents binding of quantum dots. FIG. 13A and left panel of FIG. 13B show copper that has been masked on one side under a 655 nm fluorescence filter. The right panel of FIG. 13B is a white light image of the left panel.

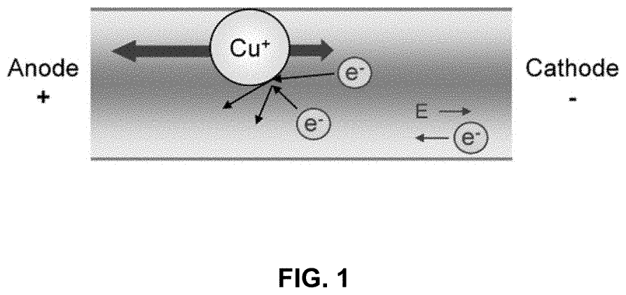

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of”.

As used in the specification and the appended claims, the singular forms “a,” “an” and “the” include plural referents unless the context clearly dictates otherwise. Thus, for example, reference to “a copper interconnect,” includes, but is not limited to, arrays or stacks of two or more such interconnects, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a solution or suspension of quantum dots refers to an amount that is sufficient to achieve the desired improvement in the property modulated by the formulation component, e.g. achieving the desired level of modulus. The specific level in terms of wt % in a composition required as an effective amount will depend upon a variety of factors including the size of the quantum dots, the functional groups attached to the quantum dots, and the size and number of nanopores in the metal surfaces upon which the quantum dots are drop cast.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

As used herein, “electromigration” is when the momentum of a moving electron is transferred to a nearby activated ion, causing the ion to move from its original position. Over time, this force displaces a significant number of atoms from their original positions.

As used herein, a “void,” sometimes also referred to as an “internal failure open circuit” is damage to a metal circuit component presenting, in some aspects, as a small hole, pit, or nanopore in the surface. In one aspect, voids can result during the formation of the Cu interconnects, from electromigration, anodization, contact with acids or etchants, or other chemical or electrochemical processes. In one aspect, the presence of voids can lead to failure of the electronic components that contain them.

A “passive component” in an electrical circuit is incapable of controlling current by means of another electrical signal. In one aspect, passive components include resistors, capacitors, inductors, interconnects, and the like.

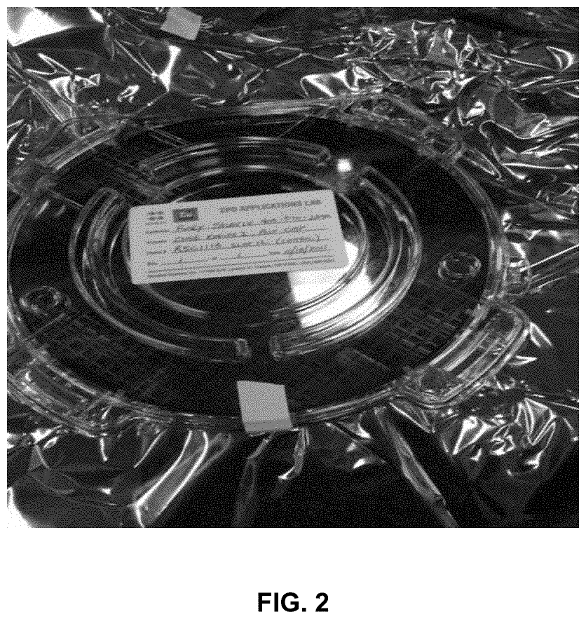

An “interconnect,” as used herein, is a component introduced into a silicon integrated circuit (e.g., as shown in FIG. 2) to improve certain aspects of performance such as, for example, by reducing propagation delays and/or power consumption. In one aspect, an interconnect can be fabricated from copper. In a further aspect, since copper is a good conductor, a copper interconnect can have smaller dimensions than one made from another material (e.g., aluminum) and requires less energy for the passage of electricity. In another aspect, a void introduced in a copper interconnect can lead to failure of the interconnect as well as of the device into which the interconnect has been incorporated.

As used herein, “failure analysis” refers to the process of identifying the cause of a failure in an electronic component. In one aspect, failure analysis may also include attempts to mitigate or prevent the causes of failure. In one aspect, the methods disclosed herein are useful during the process of failure analysis for identifying the locations of voids, cracks, pits, and other damaged sites on copper surfaces.

“Quantum dots” are semiconductor particles only a few nanometers in size. Due to their small size, quantum dots have different optical and/or electronic properties compared to larger particles. In one aspect, quantum dots can be illuminated by UV light, causing excitation of an electron from the valence band to the conductance band of the quantum dot. Further in this aspect, when the excited electron drops back into the valence band, it releases energy by emitting light. This emission can be observed as photoluminescence. Emission wavelength of quantum dots depends on both their size and their composition.

As used herein, “drop casting” or “dropcasting” refers to the formation of a thin layer on a surface by dropping a solution or suspension (e.g., of quantum dots) on the surface and evaporating the solvent. In one aspect, the methods herein employ quantum dots that have been drop cast on copper surfaces.

### Method for Detecting Defects on a Metal Surface

In one aspect, provided herein is a method for detecting defects on a metal surface, the method including the following steps:
a. providing a metal surface;
b. contacting the metal surface with quantum dots, wherein the quantum dots have been functionalized with a chemical group and wherein the quantum dots localize to areas of damage on the metal surface;
c. optionally, rinsing the metal surface; and
d. visualizing the quantum dots on the metal surface.

In one aspect, the method disclosed herein is useful in detecting any type of damage or defect that commonly occurs on a metal surface including nanopores, voids, cavities, cracks, pits, and combinations thereof. In one aspect, the damage or defect has a diameter of 1000 nm or less, or has a diameter of from about 1 nm to about 1000 nm, or has a diameter of about 1, 5, 10, 25, 50, 75, 100, 150, 200, 250, 300, 350, 400, 450, 500, 550, 600, 650, 700, 750, 800, 850, 900, 950, or about 1000 nm, or a combination of any of the foregoing values, or a range encompassing any of the foregoing values.

### Metal Surface Characteristics

In one aspect, the method disclosed herein is useful for detecting defects on a copper surface. In some aspects, the copper surface can be an electronic component or part of an electronic component, including, but not limited to, a copper interconnect, another copper contact between two elements, or a copper wire. In one aspect, the copper surface is a copper interconnect. In an alternative aspect, the copper surface has a thickness of less than about 0.25 mm, less than about 0.5 mm, less than about 0.75 mm, less than about 1.0 mm, or a combination of any of the foregoing values or range encompassing any of the foregoing values. In a further aspect, the metal surface has a surface area of from about 50 mm² to about 250 mm², or of about 50, 100, 150, 200, or 250 mm², or a combination of the foregoing values, or a range encompassing the foregoing values. In one aspect, the surface area is 150 mm² and has dimensions of 15 mm by 10 mm. In another aspect, the methods disclosed herein are not limited by surface area and can be used on a surface larger or smaller than those previously described. In a still further aspect, multiple distinct metal surfaces can be visualized by the method disclosed herein at the same time, provided they are in close proximity to one another (e.g., an array of copper elements that are part of the same electronic device).

### Quantum Dots

In one aspect, quantum dots useful herein include quantum dots incorporating any of the following semiconductor materials: AlN, CdS (hexagonal phase), CdS (cubic phase), CdSe (hexagonal phase), CdSe (cubic phase), CdTe, GaN, PbS, PbSe, TiO₂, ZnS, ZnO, InGaP, and combinations thereof, including, but not limited to, combinations where one material forms a core and a second material forms a shell around the core. In one aspect, a CdSe core (of any crystal structure) is surrounded by a ZnS outer shell.

In one aspect, the method disclosed herein uses commercially available quantum dots such as, for example, CdSe/ZnS quantum dots. In a further aspect, CdSe/ZnS quantum dots include a spherical core of CdSe that can be capped with an epitaxial ZnS shell. In one aspect, the quantum dots include an amphiphilic polymer coating. Further in this aspect, adjacent to the epitaxial ZnS shell is a monolayer of oleic acid/octadecylamine and a further monolayer of amphiphilic polymer. In one aspect, it is the amphiphilic polymer layer that is functionalized. In an alternative aspect, the quantum dots include a surface layer that can be trioctylphosphine oxide or a similar compound. In one aspect, the surface layer is hydrophobic.

In some aspects, the quantum dots are at least partially covered by a functionalizable amphiphilic polymer coating outer shell, a surface hydrophobic layer, or a combination thereof.

In a further aspect, when quantum dots bearing a positively charged functional group such as, for example, an amino group, are drop cast on the metal surface, they do not bind to damage sites, even when their size and composition is otherwise identical to the negatively charged quantum dots that do bind to the metal surface.

In one aspect, the chemical group with which the quantum dots are functionalized has a negative charge. Further in this aspect, the chemical group can be a carboxyl group. In another aspect, the quantum dots have a total diameter of from about 5 nm to about 25 nm, or about 5, 6, 7, 8, 9, 10, 11, 12, 13, 14, 15, 16, 17, 18, 19, 20, 21, 22, 23, 24, or 25 nm, or a combination of any of the foregoing values, or a range encompassing any of the foregoing values. In a further aspect, the total diameter is from about 7 nm to about 20 nm. In one aspect, the total diameter of the optically active region of the quantum dots is from about 5 to about 10 nm, or is about 5, 6, 7, 8, 9, or 10 nm, or a combination of any of the foregoing values, or a range encompassing any of the foregoing values. In some aspects, the optically active region is coated with outer passivating layers to reach the total diameter described above.

In another aspect, the quantum dots can be functionalized in numerous ways depending upon the liquid in which the quantum dots are suspended. In some aspects, the quantum dots can be suspended in a hydrophobic layer on a surface.

In one aspect, binding depends only weakly on the diameter of the quantum dots. However, in another aspect, the diameter of the quantum dots determines their color (i.e., emission wavelength).

### Rinsing Step

In one aspect, the quantum dots are placed on the metal surface using drop casting or another technique. In a further aspect, the surface is optionally allowed to dry following drop casting. In one aspect, the quantum dots are commercially supplied in aqueous solution or suspension. In one aspect, the aqueous solution is diluted to about 10 μM with deionized water prior to drop casting. In one aspect, the quantum dots are provided in a colloidal suspension in water with a concentration of from about 1 nM to about 100 μM, or about 1 nM, 5 nM, 10 nM, 50 nM, 100 nM, 500 nM, 1 μM, 5 μM, 10 μM, 50 μM, 100 μM, or a combination of any of the foregoing values, or a range encompassing any of the foregoing values. In one aspect, the quantum dots are provided as a colloidal suspension of quantum dots in water with a concentration of approximately 10 μM.

In another aspect, the concentration of quantum dots needed for sufficient binding scales inversely with incubation time. In a further aspect, incubation time for quantum dot binding is from about 2 to about 12 hours, or is about 2, 3, 4, 5, 6, 7, 8, 9, 10, 11, or about 12 hours, or a combination of any of the foregoing values, or a range encompassing any of the foregoing values. In a further aspect, for a shorter incubation time such as, for example, 2 hours, a higher concentration of quantum dots (for example, about 10 μM) can be used, whereas for a longer incubation time such as, for example, 12 hours, a lower concentration of quantum dots (for example, about 10 μM) can be used.

In a further aspect, after application of the quantum dots and optional drying step, the metal surface is optionally rinsed from 1 to 5 times, or 1, 2, 3, 4, or 5 times, or a combination of any of the foregoing values, or a range encompassing any of the foregoing values. In a further aspect, the surface is rinsed with deionized water. In a still further aspect, the rinsing is carried out at room temperature. In any of these aspects, rinsing with deionized water serves to remove unbound quantum dots from the metal surface but does not remove quantum dots bound to sites of defects or damage.

### Detection Methods

In one aspect, when quantum dots have bound to a metal surface bearing damage or defects, the quantum dots cause a change in the optical properties of the surface. In a further aspect, this change in optical properties can be visualized by any technique known in the art such as, for example, fluorescence detection, X-ray detection, optical microscopy, atomic force microscopy, or a combination thereof. In a further aspect, the quantum dots bound to the metal surface have a fluorescence emission wavelength of about 450 nm to about 665 nm, or of about 450, 490, 525, 530, 540, 550, 560, 570, 590, 580, 600, 610, 620, 630, 640, 645, 650, 655, 660, or about 665 nm, or a combination of any of the foregoing values, or a range encompassing any of the foregoing values. In one aspect, the quantum dots have a fluorescence emission wavelength of about 655 nm.

In one aspect, provided herein is a method for detecting nanometer-scale damage on a copper interconnect, the method comprising drop casting carboxyl-functionalized quantum dots on the copper interconnect, optionally drying the copper interconnect, rinsing the copper interconnect at least four times with deionized water, and visualizing the quantum dots under a 665 nm fluorescence filter. Further in this aspect, the quantum dots localize to the sites of any damage on the copper interconnect.

In one aspect, the method disclosed herein can be used to detect nanopores from about 10 nm to about 1000 nm in diameter, or about 10, 20, 50, 100, 150, 200, 250, 300, 350, 400, 450, 500, 550, 600, 650, 700, 750, 800, 850, 900, 950, or about 1000 nm, or a combination of the foregoing values, or a range encompassing the foregoing values. In another aspect, the method disclosed herein can be used to detect nanopores from about 20 nm to about 50 nm in diameter.

### Method for Removing Bound Quantum Dots from a Surface

In a further aspect, provided herein is a method for removing bound quantum dots from a metal surface, the method including the following steps:
a. heating the metal surface to a first temperature for a first time period, wherein the first temperature is higher than room temperature;
b. allowing the surface to cool to room temperature; and
c. wiping the quantum dots from the surface using a lint-free tissue, cloth, or wipe.

Further in this aspect, the first temperature is greater than or equal to 50° C., or is about 50, 100, 150, 175, 200, 225, 250, 275, about 300° C., or a combination of any of the foregoing values, or a range encompassing any of the foregoing values. In one aspect, the first temperature is about 200° C. In an alternative aspect, the first temperature is 100° C.

In another aspect, the first time period is from about 20 minutes to about 60 minutes, or is 20, 30, 40, 50, or about 60 minutes, or a combination of any of the foregoing values, or a range encompassing any of the foregoing values. In one aspect, the first time period is about 30 minutes.

In one aspect, the metal surface is heated to about 100° C. for about 30 minutes.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Materials

99.99% pure copper foil of various thicknesses (primarily 0.5 mm) was purchased from Sigma-Aldrich and anodized as described below. In some experiments, copper foil was cut to 15 mm length by 10 mm width. Negatively charged, carboxy-functionalized CdSe/ZnS 650 NanoCrystals (NC) quantum dots suspended in water at a concentration of 1 mg/mL were obtained from Sigma-Aldrich and were diluted and used for drop casting. These quantum dots were coreshell-type quantum dots as described previously and have a characteristic wavelength of 655 nm. Positively charged, amino-functionalized CdSe/ZnS 650 NanoCrystals (NC) quantum dots suspended in water at a concentration of 1 mg/mL were obtained from Sigma-Aldrich and were diluted and used for drop casting. These quantum dots were coreshell-type quantum dots as described previously and have a characteristic wavelength of 655 nm.

### Example 2: Anodization and Masking of Copper

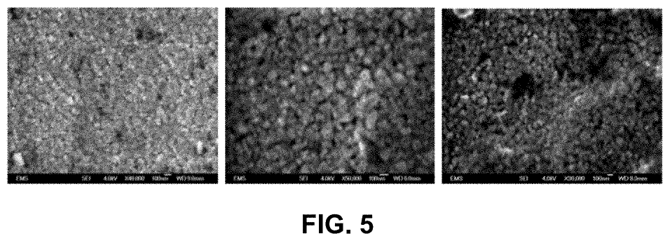

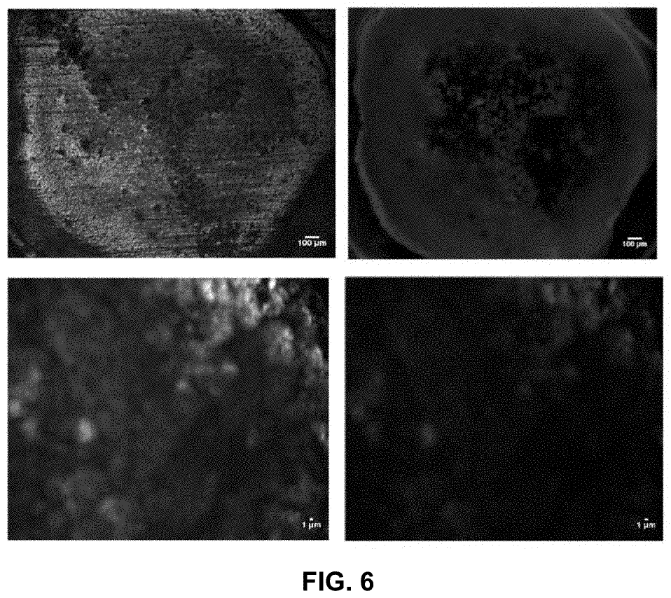

Copper foil surfaces were anodized in 3.5 M sodium hydroxide solution at 10° C. to produce nanopores in the surfaces. The anode was the copper sample and a platinum electrode was used as a cathode. A voltage of 40V was applied for 600 seconds, 900 seconds, and/or 1500 seconds. Anodized copper surfaces were examined under a JEOL 6320 Field Emission Scanning Electron Microscope (FESEM). SEM micrographs of the nanopores produced under each set of conditions can be seen in FIG. 5. Average nanopore size was about 25 nm on a typical 1×10 mm surface area sample of 0.5 mm thick copper foil.

In some experiments, a portion of the copper surface was masked with tape to prevent anodization of that portion.

### Example 3: Interaction of Carboxyl-Functionalized Quantum Dots with Damaged and/or Anodized Copper Surfaces

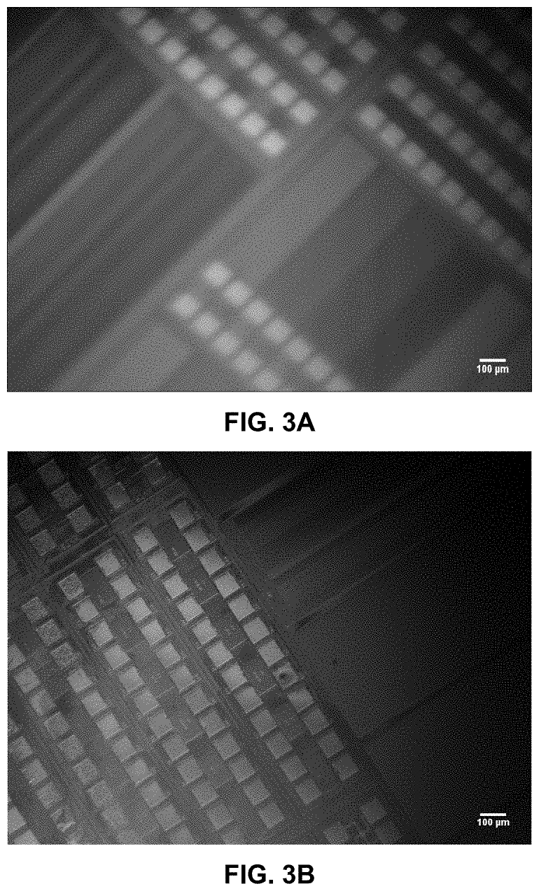

Anodized surfaces were prepared as in Example 2. In some experiments, copper surfaces containing voids, cracks, pits, or other damage were used without further treatment. Images of sample copper surfaces are shown in FIGS. 3A-3B.

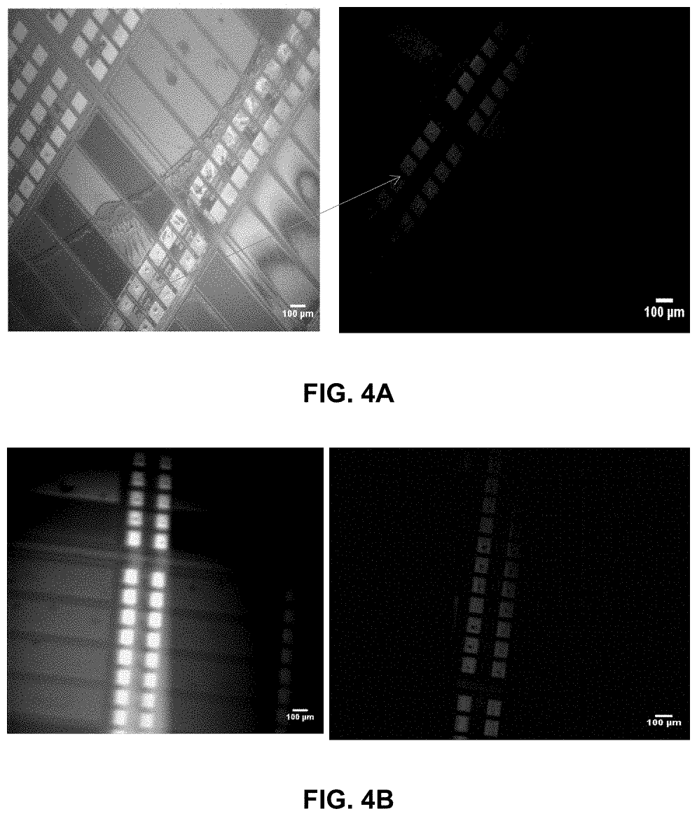

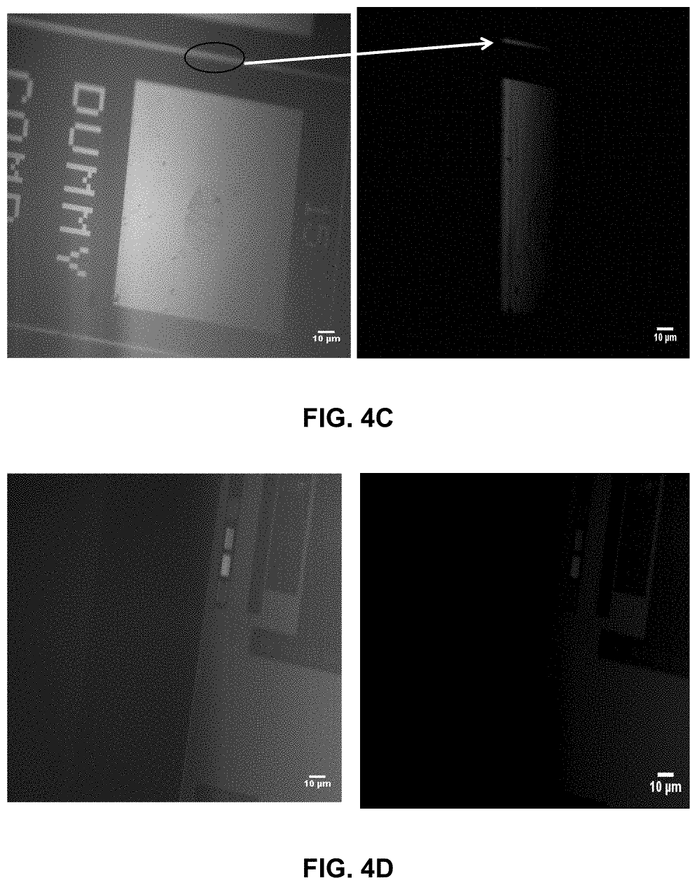

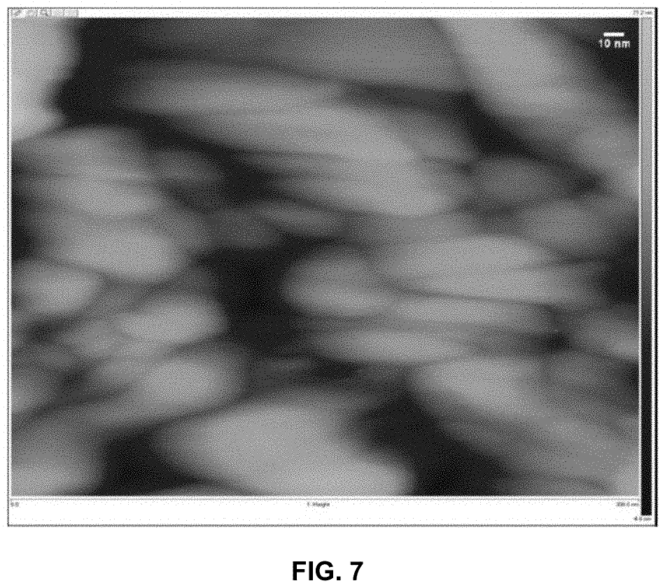

A solution containing carboxyl-functionalized CdSe/ZnS quantum dots was diluted to 10 μM and drop cast on the copper surfaces. After drop casting, the surfaces were left overnight (see FIG. 6) and quickly washed 4 times with deionized water at room temperature. After each wash, surfaces were then visualized under white light or under a 655 nm fluorescence filter at 4× and 50× magnification (FIGS. 4A-D). Bound quantum dots at damage sites (i.e., nanopores from anodization) exhibited red fluorescence under the 655 nm filter indicating localization to damage sites even after washing. The emitted light intensity decreased somewhat with each wash until it reached a constant minimum value after 4 washes; thus, the only quantum dots remaining on the surface after 4 washes were concluded to be the ones bound to nanopores. FIG. 7 shows an atomic force micrograph of the copper surface with bound quantum dots.

In a typical experiment, about 70% of nanopores on a copper surface were seen to bind with quantum dots, though this was somewhat dependent on the concentration of quantum dots in the drop cast solution, with higher concentrations of quantum dots resulting in a greater percentage of nanopores bound to quantum dots.

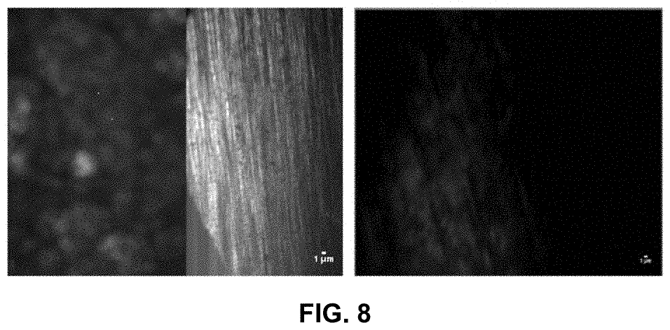

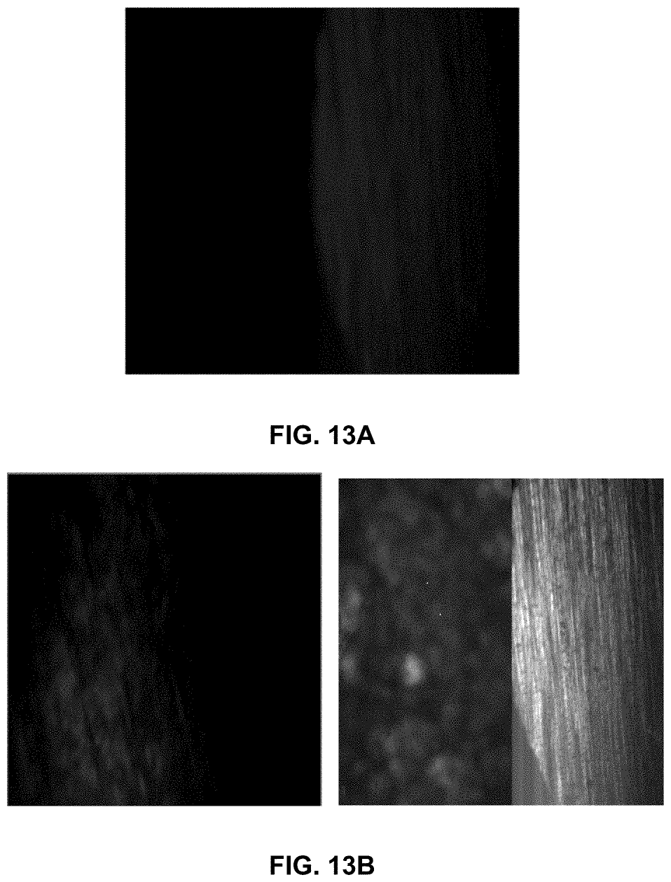

In some experiments, when a portion of the copper surface was masked to prevent anodization, quantum dots did not bind to the masked, unanodized portion of the surface after washing due to the lack of binding sites (FIG. 8, FIG. 13A-B).

### Example 4: Interaction of Amino-Functionalized Quantum Dots with Damaged and/or Anodized Copper Surfaces

Anodized surfaces were prepared as in Example 2. In some experiments, copper surfaces containing voids, cracks, pits, or other damage were used without further treatment.

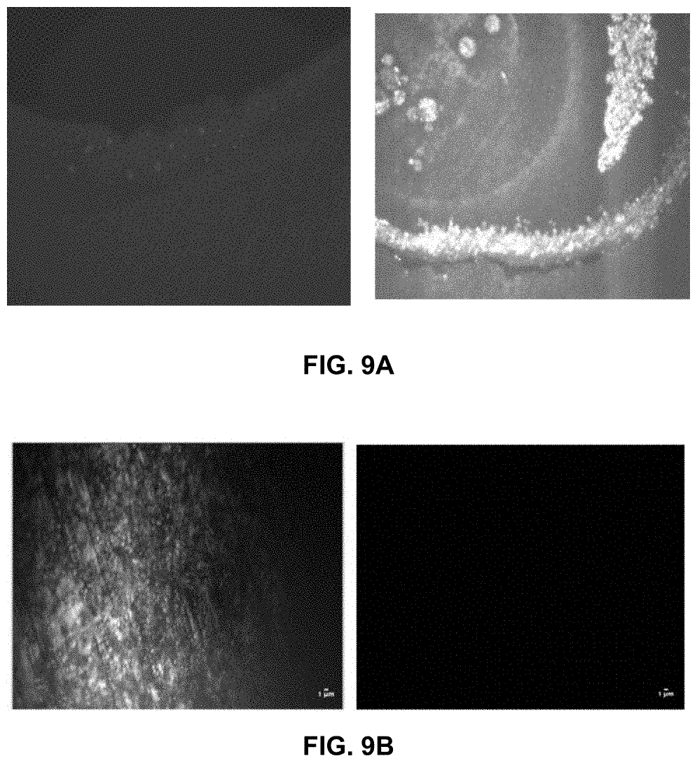

A solution containing amino-functionalized CdSe/ZnS quantum dots was diluted to 10 μM and drop cast on the copper surfaces. After drop casting, the surfaces were left overnight and washed 4 times. No amino-functionalized quantum dots were seen to bind to the anodized copper (see FIG. 9A-B).

### Example 5: Removal of Quantum Dots from Copper Surfaces

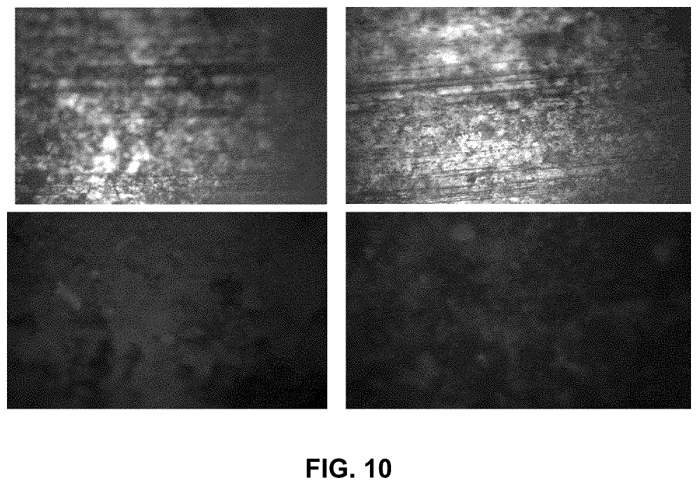

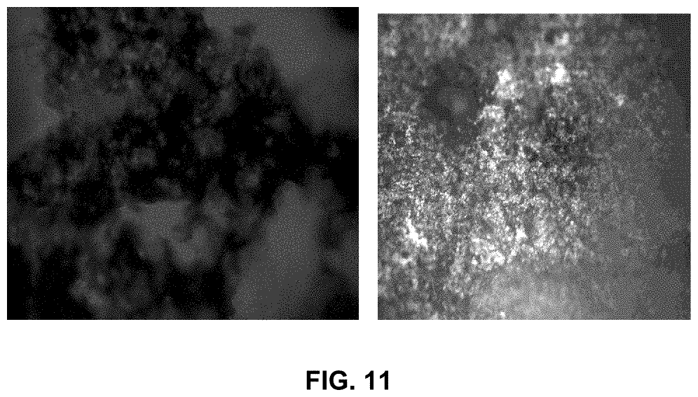

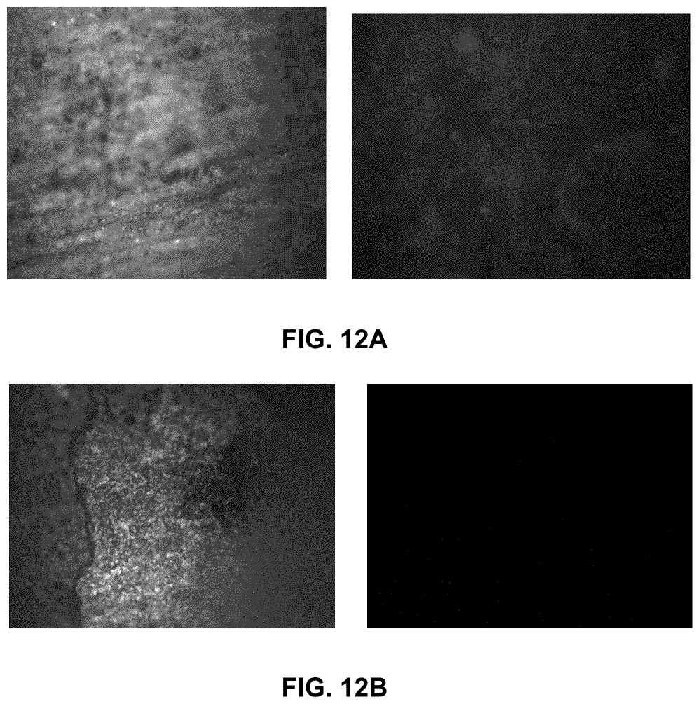

As noted in Example 3, bound carboxy-functionalized quantum dots are not removed by simple and repeated washing (FIGS. 10-11). Thus, a heating step was evaluated as a way to remove quantum dots from the copper surfaces. The anodized copper surfaces were heated to 100° C. for 30 minutes, cooled, and wiped with a lint-free cloth to remove bound quantum dots. The surfaces were then visualized with white light as well as under a 655 nm fluorescence filter, showing that bound quantum dots were removed from the surfaces with heat (FIGS. 12A-B). The lint-free cloth was also visualized under a 655 nm fluorescence filter, showing that the quantum dots had transferred to the lint-free cloth and were no longer on the copper surface.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. A method for detecting defects on a metal surface, the method comprising:
(a) providing a metal surface;
(b) contacting the metal surface with quantum dots, wherein the quantum dots have been functionalized with a chemical group and wherein the quantum dots localize to areas of damage on the metal surface;
(c) optionally, rinsing the metal surface; and
(d) visualizing the quantum dots on metal surface.

2. The method of claim 1, wherein the metal surface comprises a copper surface.

3. The method of claim 2, wherein the copper surface comprises an electronic component.

4. The method of claim 3, wherein the electronic component comprises a copper interconnect, a copper contact, or a copper wire.

5. The method of claim 4, wherein the electronic component is a copper interconnect.

6. The method of claim 2, wherein the copper surface has a thickness of less than about 1 mm.

7. The method of claim 1, wherein the quantum dots are CdSe/ZnS quantum dots.

8. The method of claim 7, wherein the quantum dots comprise a CdSe core and a ZnS shell.

9. The method of claim 7, wherein the quantum dots further comprise at least one amphiphilic layer.

10. The method of claim 9, wherein the at least one amphiphilic layer comprises a monolayer of oleic acid/octadecylamine and a monolayer of amphiphilic polymer.

11. The method of claim 1, wherein the chemical group on the quantum dots has a negative charge.

12. The method of claim 11, wherein the chemical group is a carboxyl group.

13. The method of claim 1, wherein the quantum dots have a diameter of from about 7 nm to about 20 nm.

14. The method of claim 1, wherein contacting the metal surface with quantum dots is accomplished by drop casting.

15. The method of claim 1, wherein the metal surface is rinsed with deionized water.

16. The method of claim 15, wherein the metal surface is rinsed from 1 to 5 times.

17. The method of claim 1, wherein step (d) is accomplished using fluorescence detection, X-ray detection, optical microscopy, atomic force microscopy, or a combination thereof.

18. The method of claim 17, wherein the quantum dots have a fluorescence emission wavelength of about 665 nm and are visualized using fluorescence detection.

19. The method of claim 1 wherein the defects comprise nanopores, voids, cavities, cracks, pits, or a combination thereof.

20. A method for removing bound quantum dots from a metal surface, the method comprising:
(a) heating the metal surface to a first temperature for a first time period, wherein the first temperature is higher than room temperature;
(b) allowing the surface to cool to room temperature; and
(c) wiping the quantum dots from the surface using a lint-free tissue, cloth, or wipe.

21-22. (canceled)

23. Quantum dots configured to detect at least one defect on a metal surface, the quantum dots comprising an outer shell or a surface layer.

24-27. (canceled)
